(19) United States
(12) Patent Application Publication
(10) Pub. No.: US 2017/0118965 A1
(43) Pub. Date: May 4, 2017

(54) HUMANIZED MOUSE MODELS AND USES THEREOF

(71) Applicant: Champions Biotechnology, Inc, HACKENSACK, NJ (US)

(72) Inventors: Gilson S Baia, Baltimore, MD (US); Keren Paz, Tenafly, NJ (US); David SIDRANSKY, Baltimore, MD (US)

(73) Assignee: Champions Biotechnology, Inc, HACKENSACK, NJ (US)

(21) Appl. No.: 15/319,039

(22) PCT Filed: Jun. 17, 2015
(86) PCT No.: PCT/US15/36180

§ 371 Date: Dec. 15, 2016

Related U.S. Application Data
(60) Provisional application No. 62/165,464, filed on May 22, 2015.
(60) Provisional application No. 62/013,427, filed on Jun. 17, 2014.

(51) Int. Cl. A01K 67/027; A61K 49/00

(52) U.S. Cl. CPC A01K 67/0271; A61K 49/0008; A01K 2207/15; A01K 2267/03; A01K 2227/105; A01K 2207/12; A01K 2267/0331

(57) ABSTRACT

The invention relates generally to a humanized mouse model and uses thereof. Specifically, the invention relates to methods for generating, maintaining, and expanding a culture of leukocytes in heterologous animals. This invention also relates to use of these animals as models of human immune system for testing molecules in order to treat a disease or disorder such as cancer.

### CROSS-REFERENCE TO RELATED APPLICATIONS

This application claims priority to U.S. Provisional Patent Applications 62/013,427, filed Jun. 17, 2014 and 62/165,464, filed May 22, 2015, both of which are incorporated herein by reference in their entirety.

### FIELD OF THE INVENTION

The invention relates generally to a humanized mouse model and uses thereof. Specifically, the invention relates to a humanized mouse model comprising a human tumor xenograft. The invention further relates to methods for identifying therapeutic molecules using the humanized mouse model.

The invention additionally relates to methods for generating, expanding and maintaining a culture of leukocytes in heterologous animals. The invention also relates to the use of these animals as models of human immune system for testing molecules in order to treat a disease or disorder.

### BACKGROUND OF THE INVENTION

The blockade of immune checkpoints is a promising therapeutic avenue for cancer therapy, with durable objective responses observed in patients with a variety of solid tumors. Despite these successes however, current animal models often fail to pinpoint immunotherapies with the greatest clinical potential due in part to differences between human and murine immune systems. Hence, there exists a need for reliable preclinical tools to test these drugs directly against human cancers in the context of a human immune system. To circumvent this limitation, the inventors of the instant application have developed an ImmunoGraft model, whereby two innovative technologies, the Champions TumorGraft (patient-derived xenograft) and humanized mice (immune-deficient mice reconstituted with a human immune system), are combined in a single platform.

The spleen is the largest secondary lymphoid organ containing about one-fourth of the body's lymphocytes. The splenic subsets comprise of cells of the myeloid lineage, including dendritic cells and macrophages. In addition, in rodents extra medullary hematopoiesis is also present in the spleens and a minor fraction (<1%) of human CD34+ progenitor cells can be identified in splenocyte preps of humanized mice.

Adoptive cell therapy is a therapeutic approach comprising administration of a patient's own (autologous) or donor (allogeneic) anti-tumor or anti-pathogen lymphocytes, following a lymphodepleting preparative regimen. This approach has emerged as a potentially powerful tool of controlling pathological conditions, including infections and cancers. It also allows for generation of populations of lymphocytes with desired anti-pathogen specificity, which then can be available for use in case of recurrence of the pathology. The early protocols of adoptive transfer therapy selected the cells of desired specificity (e.g. anti-tumor leukocytes) and expanded them in the tissue culture. This approach, however, has significant limitations, including clonal selection in tissue culture, requirement for expensive tissue culture maintenance facilities, and limited scale-up potential. These concerns were partially addressed through the development of in vivo adoptive transfer protocol, which used immunodeficient animals, such as mice, to generate and maintain cultures of lymphocytes specific for a pathogen of choice. In case of cancer one protocol typically involves implantation of tumors into immunodeficient recipient animal (e.g. mouse) that has been “humanized” with xenograft of human cord blood-derived CD34+ hematopoietic stem cells (HSCs). This method however is limited by the availability of CD34+ HSCs. Furthermore the presence of tumor tissue in the humanized mouse limits scale-up potential and gives rise to safety concerns, since the resulting anti-tumor leukocyte population may also contain tumor cells. Another protocol involves implantation of tumor tissue into immunodeficient mice followed by expansion and subsequent harvesting of leukocytes that were co-implanted with tumor. While this method addresses the issue of limited availability of human cord blood-derived CD34+ HSCs, it does not resolve the limited scalability and safety concerns.

Accordingly, there exists a need for an improved adoptive transfer therapy.

### SUMMARY OF THE INVENTION

In one aspect, the invention provides a method for producing humanized mouse model, the method comprising reconstituting said mouse with human CD34+ cells, thereby producing said humanized mouse model.

In another aspect, the invention provides a humanized mouse comprising a human tumor xenograft, wherein said xenograft is capable of propagating in said mouse.

In yet another aspect, the invention provides a method for producing a mouse model, the method comprising: providing a humanized mouse; and subcutaneously implanting a human tumor xenograft in said mouse, thereby producing said mouse model.

In further aspect, the invention provides a method for treating a tumor in a patient, the method comprising: providing a humanized mouse comprising a human tumor xenograft; testing one or more therapeutic agents to evaluate the effect of said agents on tumor growth inhibition in said mouse; identifying an effective therapeutic agent; and administering said effective therapeutic agent to said patient, thereby treating said tumor in said patient.

In a an additional aspect, the invention provides a method for providing a personalized treatment to treat a tumor in a patient, the method comprising: providing a humanized mouse comprising a tumor xenograft obtained from said patient; testing one or more therapeutic agents to evaluate the effect of said agents on tumor growth inhibition in said mouse; identifying an effective therapeutic agent; and administering said effective therapeutic agent to said patient in order to treat said tumor in said patient, thereby providing a personalized treatment to treat said tumor in said patient.

The present invention furthermore provides a method of maintaining and expanding a culture of human leukocytes in vivo.

In one aspect, the invention relates to a method for establishing a human immune system in a non-human mammal, the method comprising: providing an immunodeficient non-human mammal; injecting said mammal with a composition, said composition comprising human CD34+ progenitor cells or splenocytes isolated from another non-human mammal, wherein said another non-human mammal is a humanized non-human mammal.

In another aspect, the invention relates to a method for testing a therapeutic approach, the method comprising: providing an immunodeficient non-human mammal; injecting said mammal with a composition, said composition comprising human CD34+ progenitor cells or splenocytes isolated from another non-human mammal, wherein said another non-human mammal is a humanized non-human mammal; testing a therapy in said mammal; and evaluating the effect of said therapy in said mammal.

The invention further provides, in another aspect, for a method of testing a cancer therapy, the method comprising: providing an immunodeficient non-human mammal; injecting said mammal with a composition, said composition comprising human CD34+ progenitor cells or splenocytes isolated from another non-human mammal, wherein said another non-human mammal is a humanized non-human mammal; introducing a tumor tissue from a patient; administering a cancer therapy to said non-human mammal; and evaluating the effect of said therapy in said non-human mammal.

In yet another aspect, the invention provides a method for selecting one or more clinical trial participants from a pool of candidates, the method comprising: providing an immunodeficient non-human mammal; injecting said mammal with a composition, said composition comprising a candidate's human CD34+ progenitor cells; administering a therapy to said non-human mammal; and evaluating the immune response of the established human immune system.

The present invention also provides for a method for maintaining a human immune system in a non-human mammal, the method comprising: injecting a naïve immunodeficient mammal with splenocytes isolated from a humanized mouse; isolating splenocytes from said injected naïve immunodeficient mammal; and injecting said isolated splenocytes into a naïve immunodeficient mammal of a subsequent generation.

In another aspect, the invention provides a method for maintaining or expanding a culture of B and T leukocytes, the method comprising: introducing leukocytes from a heterogeneous mammal into a recipient mammal; isolating splenocytes of said recipient mammal after at least 4 weeks after the introduction of said leukocytes; injecting said splenocytes into a naïve immunodeficient mammal; isolating leukocytes from said injected mammal after at least 4 weeks post the injection; and isolating said heterogeneous mammal leukocytes from said leukocytes.

In another aspect, the invention provides for a method for producing B and T leukocytes, the method comprising: introducing leukocytes from a heterogeneous mammal into a recipient mammal; isolating splenocytes of said recipient mammal after at least 4 weeks after the introduction of said leukocytes; injecting said splenocytes into a naïve immunodeficient mammal; isolating leukocytes from said injected mammal after at least 4 weeks post the injection; and isolating said heterogeneous mammal leukocytes from said leukocytes. In yet another aspect, the invention provides for isolated B and T leukocytes produced by the method described herein.

In another aspect, the invention provides for a method for producing one or more animals, each comprising a population of heterologous leukocytes, the method comprising: introducing leukocytes from a heterogeneous mammal into a recipient mammal; isolating splenocytes of said recipient mammal after at least 4 weeks after the introduction of said leukocytes; and injecting said splenocytes into a naïve immunodeficient mammal.

Furthermore, in another aspect, present invention provides for a method for producing a model of immune system of a mammal having cancer, the method comprising: introducing a tumor tissue from a heterogeneous mammal into a recipient mammal; isolating splenocytes of said recipient mammal after at least 12 weeks after the introduction of said tumor tissue; and injecting said splenocytes into a naïve immunodeficient mammal.

In another aspect the present invention additionally provides for a pharmaceutical composition comprising B and T leukocytes, produced according to the methods described hereinabove.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

FIG. 1 illustrates a flowchart of a method for humanizing mice and its therapeutic use, according to one embodiment of the invention.

FIG. 2 presents a schematic methodology for adoptive transfer of immune cells from humanized mice. For comparison, splenocytes, bone marrow and peripheral blood monocytes (PBMCs) were used.

FIG. 3 presents a graph showing flow cytometry analysis on peripheral blood of mice reconstituted with splenocytes, bone marrow or PBMCs from a humanized NOG mouse (12 weeks post reconstitution). Overall, adoptive transfer of splenocytes generated high levels of hCD45, with a robust fraction represented by human T-cells (CD3) and B-cells (CD19). Adoptive transfer of bone marrow cells generated good hCD45 reconstitution with very poor reconstitution of T-cells. Reconstitution of PBMCs was not observed

FIG. 4A presents a graph showing flow cytometry analysis on peripheral blood of mice reconstituted with splenocytes, from a humanized NOG mouse. Overall, adoptive transfer of splenocytes generated high levels of hCD45 cells. In average, 14.7%, 32% and 60.5% of viable cells were human CD45 cells at 3, 6 and 9 weeks post reconstitution, respectively.

FIG. 4B presents a graph showing flow cytometry analysis on peripheral blood of NOG mice reconstituted with splenocytes. Immune reconstitution provided robust levels of hCD45 leucocytes, with representative subsets of CD3 T-cells, CD19 B-cells and CD56 NK-cells.

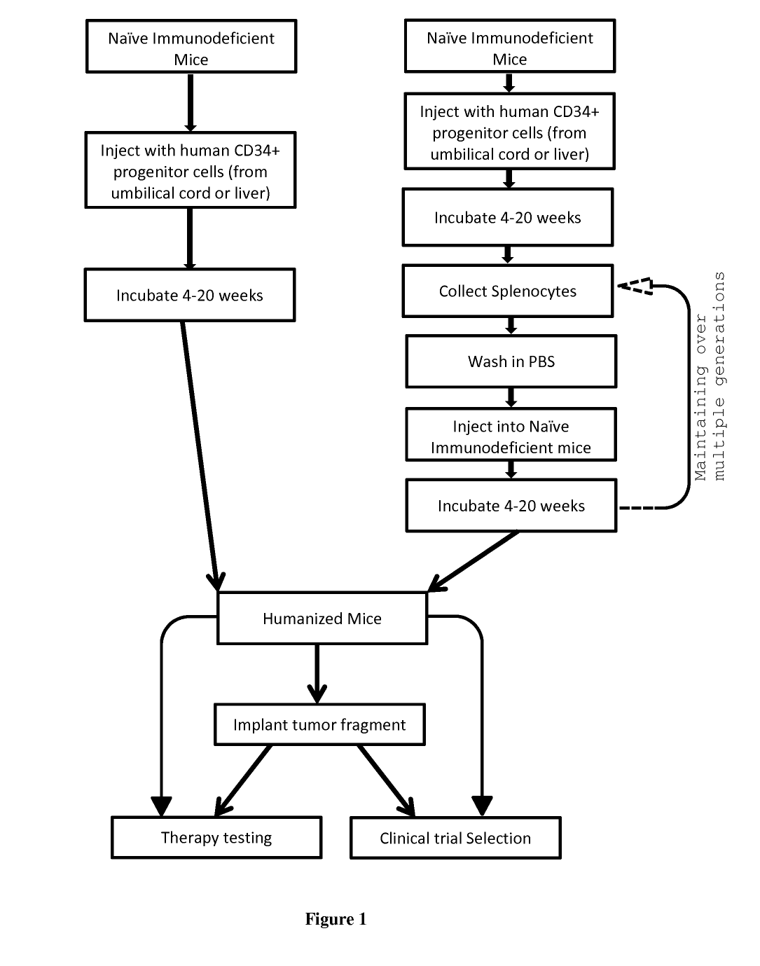

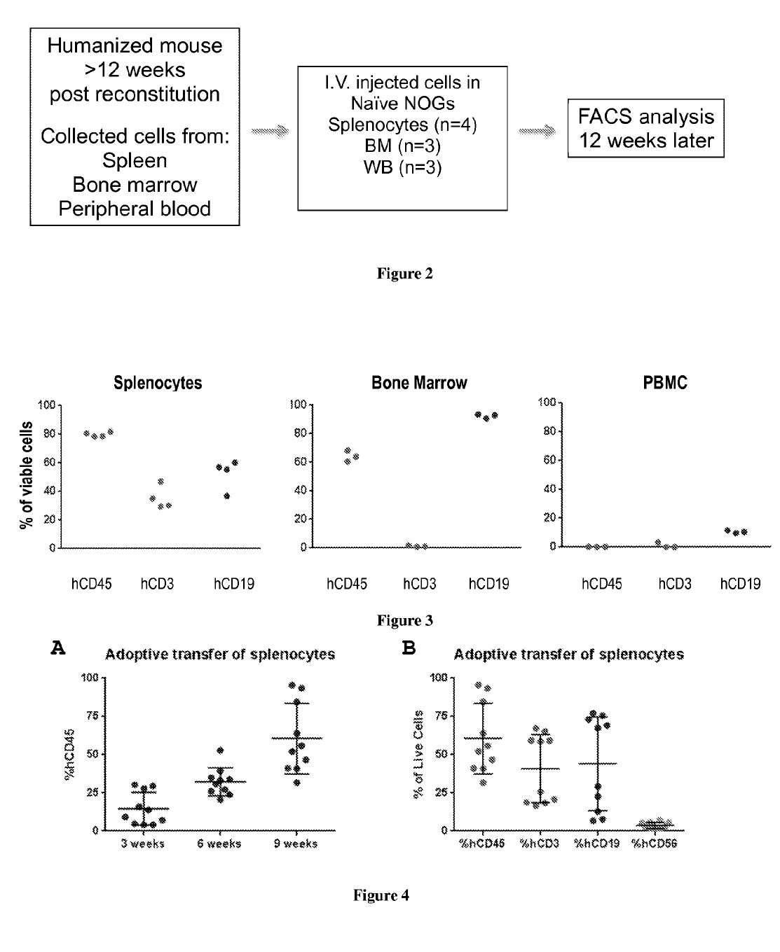

### DETAILED DESCRIPTION OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The invention provides a humanized mouse model and uses thereof. Specifically, the invention provides a humanized mouse model comprising a human tumor xenograft. The invention further provides methods for identifying therapeutic molecules using the humanized mouse model. The invention also provides methods for treating tumor and methods for providing a personalized treatment.

The inventors of the instant application have developed an ImmunoGraft mouse model, whereby two innovative technologies, the TumorGraft (patient-derived xenograft) and humanized mice (immune-deficient mice reconstituted with a human immune system), are combined in a single platform. Specifically, the inventors of the instant application have developed a humanized mouse comprising a human tumor xenograft, wherein said xenograft is capable of propagating in said mouse.

Surprisingly and unexpectedly, as demonstrated in the Example section below, tumor volumes in humanized animals are comparable to those in non-humanized animals.

The mouse can be humanized by any suitable method known to one of skilled in the art. Methods for humanizing a mouse are well known in the art and fully described in U.S. Pat. No. 8,604,271; U.S. Pat. No. 8,071,839; U.S. Pat. No. 6,676,924; U.S. Pat. No. 5,874,540; U.S. Pat. No. 8,658,154; U.S. Pat. No. 8,110,720; and U.S. Pat. No. 5,777,194 as well as U.S. Patent Application Publications US 2013/0291134; US 2013/0217043; US 2012/0066780; US 2005/0089538; and US 2002/0018750, all of which are incorporated by reference herein in their entirety.

The present invention further provides for a method of establishing and maintaining of a human immune system of in a non-human mammal. This invention also generally provides for a non-human mammal model comprising a human immune system. Specifically the present invention provides for a method of establishing a human subject's immune system in immunodeficient mice through administering isolated human CD34+ progenitor cells to said mice. This invention further provides for maintaining the human subject's immune system in immunodeficient mice through isolating splenocytes of mice previously administered with human CD34+ progenitor cells and administering the isolated splenocytes to one or more naïve immunodeficient mice. This invention additionally provides for the use of mice comprising a human subject immune system for testing therapeutic methods, specifically for testing cancer therapies.

In one embodiment, a method of the invention comprises the steps of isolating immune cells from a subject and administering the isolated cells into an immunodeficient non-human mammal thereby generating a “humanized” non-human mammal. The method of the present invention also comprises maintaining successive generations of humanized non-human mammals harboring a subject's immune cells.

The term “humanized”, as used herein refers to an immunodeficient mammal that harbors a population of heterogeneous immune cells that were introduced into it. The source of the heterogeneous immune cells may be either a donor mammal, or another humanized mammal.

The subject can be a human or a non-human mammal. Examples of non-human mammals include, but are not limited to, farm animals (e.g., cows, pigs, and horses), domesticated animals (e.g., dogs, cats, rabbits, and horses), human companion animals, zoo animals, wild animals, and laboratory animals (e.g., rats, mice, hamsters, guinea pigs, monkeys, and apes).

The methods of the invention further provide for isolation of the hematopoietic stem cells (HSCs) from the donor mammals. The methods of isolating the HSCs are well known in the art and include, for example, fluorescence activated cell sorting (FACS) targeting appropriate cellular markers. Suitable markers for each of these cell types are well known in the art, and, in case of human HSCs include CD34+, CD59+, Thy1/CD90+, C-kit/CD117+. In a preferred embodiment, the human HSCs are CD34+ HSCs. CD34+ HSC can be harvested from the subject's fetal liver, spleen or bone marrow. Each represents a separate embodiment of the invention.

This invention further provides for administration of the isolated HSCs to immunodeficient non-human mammals. HSCs can be administered to one or multiple immunodeficient mammals. Where HSCs are administered to several different immunodeficient mammals, these mammals may be of the same species or of different species to explore the effectiveness of establishing immune system in various species.

This invention further provides for the use of immunodeficient recipient non-human mammals. The recipient non-human mammals may include dogs, cats, rabbits, rats, mice, hamsters, or guinea pigs. In a preferred embodiment, the invention provides for the use of immunodeficient mice as the recipient mammals. The term “immunodeficient” as used herein refers to an animal's impaired or otherwise not fully functioning immune system, for example an inability to produce a normal amount of B-cells, T-cells, NK-cells, etc. The immunodeficient phenotype can be, in one embodiment, a result of a naturally occurring genetic defect, or, in another embodiment, a result of an induced genetic defect. Immunodeficiency may be produced by, for example, but not limited to, mutations, irradiation, a chemical or pharmaceutical, or a virus. Examples of immunodeficient mice include nude (nu⁻/nu⁻) mice, nude and severe combined immunodeficiency (SCID) mice, non obese diabetic (NOD) mice, NOD/SCID mice, NSG (NOD/SCID/γc^{-/-}) mice, Non-Obese Diabetic (NOD) Shi-Scid IL-2R γ^{null} (NOG) mice, Rag-1 (rag-1^{-/-}/γc^{-/-}) mice, or Rag-2 (rag-2^{-/-}/gc^{-/-}) BRG mice (BALB/c-Rag2^{null}/IL2rγ^{null}), Rag 1^{-/-} mice, Rag 1^{-/-}/γc^{-/-} mice, Rag 2^{-/-} mice, and Rag 2^{-/-/}γc^{-/-} mice, Scid mouse, NOD/Shi mouse, IL-2R γ^{null} mouse, NOD/Sci-Scid mouse. In a preferred embodiment, the immunodeficient mice are NOD mice carrying various mutations in the interleukin-2 receptor gamma chain (IL2Rγ) gene. Examples of such mice include NOD/SCID IL2rγ^{null} and NOD/SCID IL2rγ^{Trunc} mice. In a particularly preferred embodiment, the immunodeficient mice are NOG (Pkrdc^{scid}IL2Rγ^{tm1Sug}) mice.

After leukocytes injection into an immunodeficient mouse strain, the leukocytes migrate via the recipient's vascular system into mouse tissue, most notably the spleen and bone marrow (described in Simpson-Abelson et al., 2008, The Journal of Immunology, 180, 7009, which is incorporated herein by reference in its entirety). These cells retain their ability to differentiate and are capable of expansion after tumor injection (see Bernard et al., 2008, Clinical and Experimental Immunology, 152, 406 which is incorporated herein by reference in its entirety). Thus the invention provides for harvesting of splenocytes after the heterologous subject's immune system has been established and the leukocytes migration into spleen has taken place. An immune system can be considered “established” after it has been given an appropriate amount of time to develop in the animal after inoculation of the HSCs into the animal. One indication that immune system has been “established” is when the mouse humanized with human CD34+ HSCs is capable of providing mature leukocytes. The time allowed for the tissue for developing in the animal is referred to as an “establishment period.” In another embodiment, the establishment period is 7-15 weeks. In another embodiment, the establishment period is 8-14 weeks. In another embodiment, the establishment period is 9-13 weeks. In another embodiment, the establishment period is 10-12 weeks. In another embodiment, the establishment period is 8-15 weeks. In another embodiment, the establishment period is 9-15 weeks. In another embodiment, the establishment period is 10-15 weeks. In another embodiment, the establishment period is 12-15 weeks. In another embodiment, the establishment period is 7-15 weeks. In another embodiment, the establishment period is 13-15 weeks. In another embodiment, the establishment period is 14-15 weeks. In another embodiment, the establishment period is 6-7 weeks. In another embodiment, the establishment period is 6-8 weeks. In another embodiment, the establishment period is 6-9 weeks. In another embodiment, the establishment period is 6-10 weeks. In another embodiment, the establishment period is 6-11 weeks. In another embodiment, the establishment period is 6-12 weeks. In another embodiment, the establishment period is 6-13 weeks. In another embodiment, the establishment period is 6-14 weeks. In another embodiment, the establishment period is 8-10 weeks. In another embodiment, the establishment period is 9-11 weeks. In another embodiment, the establishment period is 10-12 weeks. In another embodiment, the establishment period is 11-13 weeks. In another embodiment, the establishment period is 12-14 weeks. In another embodiment, the establishment period is 13-15 weeks. In another embodiment, the establishment period is 7 weeks. In another embodiment, the establishment period is 8 weeks. In another embodiment, the establishment period is 9 weeks. In another embodiment, the establishment period is 10 weeks. In another embodiment, the establishment period is 11 weeks. In another embodiment, the establishment period is 13 weeks. In another embodiment, the establishment period is 14 weeks. In another embodiment, the establishment period is 15 weeks. In another embodiment, the establishment period more than 15 weeks. In a preferred embodiment, the establishment period is 12 weeks.

In some embodiments, the mouse model is reconstituted with human CD34+ cells. In one embodiment, after a pre-determined time of reconstitution (e.g., 10 weeks post hCD34+ reconstitution), the reconstituted mouse is capable of providing mature human CD45+ cells. In a particular embodiment, the reconstituted mouse is capable of providing hCD3+, hCD4+, and hCD8+ cells.

In another preferred embodiment the establishment period is determined experimentally through detection of mature leukocytes. For example detection of mature leukocytes in the recipient mammal's peripheral blood or organs such as spleen or bone marrow is indicative of immune system having been established and migration having taken place. The methods of detecting the target cells are well known in the art and include, but not limited to immunohistochemistry, fluorescent in situ hybridization (FISH), fluorescence activated cell sorting (FACS) targeting appropriate cellular markers. For example for human T cells the suitable markers comprise human CD45, CD3, CD4, CD8 and TCR, or a combination thereof; for human B cells suitable markers comprise anti-human CD45, CD19, IgM, or a combination thereof; for human myeloid cells suitable markers comprise human CD45, Mac-1, Gr-1, CD16, CD56, MHC Class II, or a combination thereof; for human NK cells suitable markers comprise human CD45, CD16, CD56, or a combination thereof; for human NKT cells suitable markers comprise CD45, CD3, CD4, CD8, CD16, CD56, or a combination thereof. Alternatively the maturation of leukocytes can be ascertained through detection of specific nucleic acids or proteins in routine biochemical assays, such as PCR or immunoblotting.

The methods of the invention provide for harvesting of leukocytes from a one or more of recipient's tissues. In one embodiment the leukocytes are harvested from the recipient's lungs. In another embodiment the leukocytes are harvested from the recipient's kidney. In another embodiment the leukocytes are harvested from the recipient's intestine. In a preferred embodiment the leukocytes are harvested from the recipient's peripheral blood. In another preferred embodiment the leukocytes are harvested from the recipient's bone marrow. In a particularly preferred embodiment the leukocytes are harvested from the recipient's spleen (splenocytes).

“Harvesting” refers to removing the organ containing the cells of interest from the host animal, such as the recipient mammal and disrupting the structure of said organ sufficiently to release individual cells. Methods of harvesting leukocytes from various organs are well known in the art. For example splenocytes can be collected through mechanical disruption of the spleen by forcing the excised spleen tissue through a cell strainer or nylon mesh followed by centrifugation (see e.g. Reeves and Reeves. 2001, Removal of Lymphoid Organs. Current Protocols in Immunology. 1:111:1.9:1.9.1-1.9.3.)

In the preferred embodiment of the invention the above method is used to harvest and optionally enrich splenocytes. The resulting cell population in one embodiment comprises subject's T cells. In another embodiment, the resulting cell population consists of subject's T cells. In another embodiment, the resulting population comprises subject's B cells. In another embodiment, the resulting population consists of subject's B cells. In another embodiment the resulting population comprises a mixture of subject's T cells and B cells. In another embodiment the resulting population consists of a mixture of subject's T cells and B cells. In yet another embodiment the resulting population comprises additional types of leukocytes.

In one embodiment, leukocytes comprise at least about 50% of cells harvested post-administration and expansion in naïve immunodeficient mammals. In another embodiment, leukocytes comprise at least about 55% of cells harvested post-administration and expansion in naïve immunodeficient mammals. In another embodiment, leukocytes comprise at least about 60% of cells harvested post-administration and expansion in naïve immunodeficient mammals. In another embodiment, leukocytes comprise at least about 65% of cells harvested post-administration and expansion in naïve immunodeficient mammals. In another embodiment, leukocytes comprise at least about 70% cells harvested post-administration and expansion in naïve immunodeficient mammals. In another embodiment, leukocytes comprise at least about 75% of cells harvested post-administration and expansion in naïve immunodeficient mammals. In another embodiment, leukocytes comprise at least about 80% of cells harvested post-administration and expansion in naïve immunodeficient mammals. In another embodiment, leukocytes comprise at least about 85% of cells harvested post-administration and expansion in naïve immunodeficient mammals. In another embodiment, leukocytes comprise at least about 90% of cells harvested post-administration and expansion in naïve immunodeficient mammals. In another embodiment, leukocytes comprise at least about 95% of cells harvested post-administration and expansion in naïve immunodeficient mammals. In another embodiment, leukocytes comprise at least about 96% of cells harvested post-administration and expansion in naïve immunodeficient mammals. In another embodiment, leukocytes comprise at least about 97% of cells harvested post-administration and expansion in naïve immunodeficient mammals. In another embodiment, leukocytes comprise at least about 98% of cells harvested post-administration and expansion in naïve immunodeficient mammals. In another embodiment, leukocytes comprise at least about 99% of cells harvested post-administration and expansion in naïve immunodeficient mammals. In another embodiment, leukocytes comprise 100% of cells harvested post-administration and expansion in naïve immunodeficient mammals.

In one embodiment, T cells comprise at least about 5% of leukocytes present in cell cultures harvested post-administration and expansion in naïve immunodeficient mammals. In another embodiment, T cells comprise at least about 10% of leukocytes present in cell cultures harvested post-administration and expansion in naïve immunodeficient mammals. In another embodiment, T cells comprise at least about 15% of leukocytes present in cell cultures harvested post-administration and expansion in naïve immunodeficient mammals. In another embodiment, T cells comprise at least about 20% of leukocytes present in cell cultures harvested post-administration and expansion in naïve immunodeficient mammals. In another embodiment, T cells comprise at least about 25% of leukocytes present cell cultures in harvested post-administration and expansion in naïve immunodeficient mammals. In another embodiment, T cells comprise at least about 30% of leukocytes present in cell cultures harvested post-administration and expansion in naïve immunodeficient mammals. In another embodiment, T cells comprise at least about 35% of leukocytes present in cell cultures harvested post-administration and expansion in naïve immunodeficient mammals. In another embodiment, T cells comprise at least about 40% of leukocytes present in cell cultures harvested post-administration and expansion in naïve immunodeficient mammals. In another embodiment, T cells comprise at least about 46% of leukocytes present in cell cultures harvested post-administration and expansion in naïve immunodeficient mammals. In another embodiment, T cells comprise at least about 50% of leukocytes present in cell cultures harvested post-administration and expansion in naïve immunodeficient mammals.

In one embodiment, B cells comprise at least about 5% of leukocytes present in cell cultures harvested post-administration and expansion in naïve immunodeficient mammals. In another embodiment, B cells comprise at least about 10% of leukocytes present in cell cultures harvested post-administration and expansion in naïve immunodeficient mammals. In another embodiment, B cells comprise at least about 15% of leukocytes present in cell cultures harvested post-administration and expansion in naïve immunodeficient mammals. In another embodiment, B cells comprise at least about 20% of leukocytes present in cell cultures harvested post-administration and expansion in naïve immunodeficient mammals. In another embodiment, B cells comprise at least about 25% of leukocytes present in cell cultures harvested post-administration and expansion in naïve immunodeficient mammals. In another embodiment, B cells comprise at least about 30% of leukocytes present in cell cultures harvested post-administration and expansion in naïve immunodeficient mammals. In another embodiment, B cells comprise at least about 35% of leukocytes present in cell cultures harvested post-administration and expansion in naïve immunodeficient mammals. In another embodiment, B cells comprise at least about 40% of leukocytes present in cell cultures harvested post-administration and expansion in naïve immunodeficient mammals. In another embodiment, B cells comprise at least about 46% of leukocytes present in cell cultures harvested post-administration and expansion in naïve immunodeficient mammals. In another embodiment, B cells comprise at least about 50% of leukocytes present in cell cultures harvested post-administration and expansion in naïve immunodeficient mammals. In another embodiment, B cells comprise at least about 55% of leukocytes present in cell cultures harvested post-administration and expansion in naïve immunodeficient mammals. In another embodiment, B cells comprise at least about 60% of leukocytes present in cell cultures harvested post-administration and expansion in naïve immunodeficient mammals. In another embodiment, B cells comprise at least about 65% of leukocytes present in cell cultures harvested post-administration and expansion in naïve immunodeficient mammals. In another embodiment, B cells comprise at least about 70% of leukocytes present in cell cultures harvested post-administration and expansion in naïve immunodeficient mammals.

In one embodiment, the T cells harvested post administration and expansion are CD3⁺CD8⁺ T cells. In another embodiment, the harvested T cells are CD3⁺CD4⁺ T cells. In another embodiment, harvested T cells are CD45RO⁺ memory T cells. In another embodiment, harvested T cells are CD11a⁺ memory T cells. In another embodiment harvested T cells are CXCR3⁺ memory T cells. In another embodiment, harvested T cells are CD44⁺ memory T cells. In another embodiment, harvested T cells are CD69⁻ memory T cells. In another embodiment, harvested T cells are CD69L⁻ memory T cells. In another embodiment, harvested T cells are CD25⁻ memory T cells. In another embodiment, harvested T cells are CD4⁺ FOXP3⁺ regulatory T cells (T_{reg}). In another embodiment, harvested T cells are CD4⁺ FOXP3⁻ regulatory T cells (T_{reg}). In another embodiment the harvested T cells comprise a mixture of some or all types of T cells described above.

In one embodiment, the B cells harvested post administration and expansion are CD19⁺CD20⁺ B cells. In another embodiment, harvested B cells are CD78⁺ CD138⁺ plasma cells. In another embodiment, harvested B cells are CD27⁺ memory B cells. In another embodiment, harvested B cells are CD20⁺CD27⁺CD43⁺CD70⁻ B-1 cells. In another embodiment the harvested B cells comprise a mixture of some or all types of B cells described above.

In some embodiments the leukocytes can be further enriched or isolated from the pool of harvested cells using flow cytometry, such as FACS. This technique has the advantage of being able to simultaneously isolate phenotypically pure populations of viable leukocytes for molecular analysis and subsequent use. Thus different subsets leukocytes can be isolated and analyzed for activation status, anti-tumor activity, and drug resistance.

The harvested splenocytes may be also propagated in in vitro culture. The methods of culturing splenocytes are well known in the art. Furthermore, the present invention also contemplates additional manipulation of harvested splenocytes, such as stimulation with human or non-human cytokines or antigens, or genetic manipulation such as modulating activity of endogenous genes through well-known techniques, or introducing heterologous genes into splenocytes using methods that are well known in the art.

“Enriched”, as in an enriched population of cells, can be defined based upon the increased number of cells having a particular marker in a fractionated set of cells as compared with the number of cells having the marker in the unfractionated set of cells.

“Isolated” refers to a cell that is removed from its natural environment (such as in a solid tumor) and that is isolated or separated, and is at least about 75% free, and most preferably about 90% free, from other cells with which it is naturally present, but which lack the marker based on which the cells were isolated.

The present invention furthermore provides for cryopreservation of harvested recipient splenocytes or enriched leukocytes. The methods of splenocytes cryopreservation are well known in the art (see e.g. Gad et al., 2013, Journal for ImmunoTherapy of Cancer 1 (Suppl 1), 211). The present invention contemplates numerous uses of cryopreserved tumor-associated leukocytes, including, but not limited to administration to a naïve immunodeficient mammal as described below, or in treatment of metastatic disease.

The present invention further provides for administering the splenocytes harvested from humanized mammal or enriched leukocytes to a naïve immunodeficient mammal. The naïve immunodeficient mammal can be chosen for a particular application, and can be any suitable mammal known to one of skill for the particular application. In a preferred embodiment, the recipient mammal is a mouse. In some embodiments the naïve immunodeficient mammal is the same species as the humanized mammal from which splenocytes were isolated. In some embodiments the naïve immunodeficient mammal is a different species than the humanized mammal from which splenocytes were isolated. In another embodiment, the naïve immunodeficient mammal is the same species as the subject. In another embodiment, the naïve immunodeficient mammal is a different species than the subject. In one embodiment the administering the splenocytes harvested from humanized mammal or enriched leukocytes are administered to multiple naïve immunodeficient mammals, thereby expanding of the in vivo culture of subject's leukocytes.

The invention provides for administration of a fixed number of harvested recipient mammal splenocytes or enriched leukocytes to the naïve immunodeficient mammal. In one embodiment, at least about 10⁵ cells are administered to a naïve immunodeficient mammal. In another embodiment, at least about 2×10⁵ cells are administered to a naïve immunodeficient mammal. In another embodiment, at least about 3×10⁵ cells are administered to a naïve immunodeficient mammal. In another embodiment, at least about 4×10⁵ cells are administered to a naïve immunodeficient mammal. In another embodiment, at least about 5×10⁵ cells are administered to a naïve immunodeficient mammal. In another embodiment, at least about 6×10⁵ cells are administered to a naïve immunodeficient mammal. In another embodiment, at least about 7×10⁵ cells are administered to a naïve immunodeficient mammal. In another embodiment, at least about 8×10⁵ cells are administered to a naïve immunodeficient mammal. In another embodiment, at least about 9×10⁵ cells are administered to a naïve immunodeficient mammal. In another embodiment, at least about 10⁶ cells are administered to a naïve immunodeficient mammal. In another embodiment, at least about 1.2×10⁶ cells are administered to a naïve immunodeficient mammal. In another embodiment, at least about 1.4×10⁶ cells are administered to a naïve immunodeficient mammal. In another embodiment, at least about 1.5×10⁶ cells are administered to a naïve immunodeficient mammal. In another embodiment, at least about 1.6×10⁶ cells are administered to a naïve immunodeficient mammal. In another embodiment, at least about 1.8×10⁶ cells are administered to a naïve immunodeficient mammal. In another embodiment, at least about 2×10⁶ cells are administered to a naïve immunodeficient mammal. In another embodiment, at least about 2.2×10⁶ cells are administered to a naïve immunodeficient mammal. In another embodiment, at least about 2.4×10⁶ cells are administered to a naïve immunodeficient mammal. In another embodiment, at least about 2.5×10⁶ cells are administered to a naïve immunodeficient mammal. In some embodiments, the number of administered cells is determined from the wait of the naïve immunodeficient mammal.

The invention further provides for washing of harvested splenocytes or enriched leukocytes prior to administration into naïve immunodeficient mammal. Washing solutions comprise saline, serum-free culture medium or any other solution that may be deemed suitable by a skilled artisan.

The invention further provides for expansion of the in vivo culture of leukocytes in the naïve immunodeficient mammals post-administration. In one embodiment this is achieved through administering harvested recipient mammal splenocytes or enriched leukocytes to multiple naïve immunodeficient mammals. In one embodiment, harvested recipient mammal splenocytes or enriched leukocytes are administered to 2 naive immunodeficient mammals. In another embodiment, harvested recipient mammal splenocytes or enriched leukocytes are administered to 3 naive immunodeficient mammals. In another embodiment, harvested recipient mammal splenocytes or enriched leukocytes are administered to 4 naive immunodeficient mammals. In another embodiment, harvested recipient mammal splenocytes or enriched leukocytes are administered to 5 naive immunodeficient mammals. In another embodiment, harvested recipient mammal splenocytes or enriched leukocytes are administered to 6 naive immunodeficient mammals. In another embodiment, harvested recipient mammal splenocytes or enriched leukocytes are administered to 7 naive immunodeficient mammals. In another embodiment, harvested recipient mammal splenocytes or enriched leukocytes are administered to 8 naive immunodeficient mammals. In another embodiment, harvested recipient mammal splenocytes or enriched leukocytes are administered to 9 naive immunodeficient mammals. In another embodiment, harvested recipient mammal splenocytes or enriched leukocytes are administered to 10 naive immunodeficient mammals. In another embodiment, harvested recipient mammal splenocytes or enriched leukocytes are administered to more than 10 naive immunodeficient mammals.

In another embodiment the expansion of the culture of tumor associated leukocytes in the naïve immunodeficient mammals post-administration is achieved through extending the time between administration and subsequent harvesting. In another embodiment, the expanded cultures are harvested 7-15 weeks post administration. In another embodiment, the expanded cultures are harvested 8-14 weeks post administration. In another embodiment, the expanded cultures are harvested 9-13 weeks post administration. In another embodiment, the expanded cultures are harvested 10-12 weeks post administration. In another embodiment, the expanded cultures are harvested 8-15 weeks post administration. In another embodiment, the expanded cultures are harvested 9-15 weeks post administration. In another embodiment, the expanded cultures are harvested 10-15 weeks post administration. In another embodiment, the expanded cultures are harvested 12-15 weeks post administration. In another embodiment, the expanded cultures are harvested 7-15 weeks post administration. In another embodiment, the expanded cultures are harvested 13-15 weeks post administration. In another embodiment, the expanded cultures are harvested 14-15 weeks post administration. In another embodiment, the expanded cultures are harvested 6-7 weeks post administration. In another embodiment, the expanded cultures are harvested 6-8 weeks post administration. In another embodiment, the expanded cultures are harvested 6-9 weeks post administration. In another embodiment, the expanded cultures are harvested 6-10 weeks post administration. In another embodiment, the expanded cultures are harvested 6-11 weeks post administration. In another embodiment, the expanded cultures are harvested 6-12 weeks post administration. In another embodiment, the expanded cultures are harvested 6-13 weeks post administration. In another embodiment, the expanded cultures are harvested 6-14 weeks post administration. In another embodiment, the expanded cultures are harvested 8-10 weeks post administration. In another embodiment, the expanded cultures are harvested 9-11 weeks post administration. In another embodiment, the expanded cultures are harvested 10-12 weeks post administration. In another embodiment, the expanded cultures are harvested 11-13 weeks post administration. In another embodiment, the expanded cultures are harvested 12-14 weeks post administration. In another embodiment, the expanded cultures are harvested 13-15 weeks post administration. In another embodiment, the expanded cultures are harvested 7 weeks post administration. In another embodiment, the expanded cultures are harvested 8 weeks post administration. In another embodiment, the expanded cultures are harvested 9 weeks post administration. In another embodiment, the expanded cultures are harvested 10 weeks post administration. In another embodiment, the expanded cultures are harvested 11 weeks post administration. In another embodiment, the expanded cultures are harvested 13 weeks post administration. In another embodiment, the expanded cultures are harvested 14 weeks post administration. In another embodiment, the expanded cultures are harvested 15 weeks post administration. In another embodiment, the establishment period more than 15 weeks post administration. In a preferred embodiment, the expanded cultures are harvested 12 weeks post administration.

The present invention can be used for treating any disease or disorder. In one aspect, the humanized non-human mammal of the invention can used for screening therapies targeting any disease or disorder. In another aspect, the present invention can be used to assess impact of a candidate therapy on an individual patient's immune system.

The present invention provides for a model of immune system of a mammal having cancer comprising a naïve immunodeficient mammal administered with a culture of leukocytes as described above. These models may can be used in determining the effect of a drug or treatment on the immune system of the subject that is the source of the tumor. For example the naïve mammals administered tumor associated leukocytes can be subjected to various treatment regimens and the impact on these leukocytes can be monitored. Use of naïve immunodeficient mammals for this purpose recapitulates the immune system of a cancer patient in a cancer-free background allowing for longer test regimens. Moreover, availability of several mammals that recapitulate a patient's immune system enables testing of several treatment regimens in parallel.

The term “cancer” refers to a proliferative disorder associated with unrestrained cell growth, uncontrolled cell proliferation, and decreased cell death via apoptosis. The term “tumor” is used herein to refer to a group of cells that exhibit abnormally high levels of growth and proliferation. A tumor may be malignant, pre-malignant, or; benign; malignant tumor cells are cancerous. The term “tumor” as used herein also refers to a portion of a tumor; for example a sample of a tumor. The term “tumor” as used herein also to refer to both primary tumors and metastases. The term “tumor growth” is used herein to refer to proliferation or growth by a cell or cells that comprise a tumor that leads to a corresponding increase in the size of the tumor. As used throughout, the terms “cancer” and “tumor” may in certain embodiments be used interchangeably, having all the same meanings and qualities.

According to this invention the heterologous tumor, can be a malignant tumor. The heterologous tumor, can also be, a benign tumor. In some cases, benign tumors may represent significant clinical problems and/or may behave like malignant tumors. Examples of such benign tumors include but are not limited to pituitary neurofibromas, neuromas, adenomas, and/or meningiomas. As contemplated by this invention, the heterologous tumor is a solid tumor. In some embodiments, the tumor is a portion of a tumor. Examples of solid tumors include, but are not limited to brain tumors, myeloblastomas, breast tumors, lymphomas, non-Hodgkin's lymphomas, head and neck tumors, bladder tumors, eye tumors, thyroid tumors, salivary gland tumors, adrenal tumors, esophageal tumors, intestinal tumors, gastric tumors, colon tumors, colorectal tumors, lung tumors, liver tumors, pancreatic tumors, kidney tumors, prostate tumors, muscular tumors, osseous tumors, skin tumors, and stromal/sarcoma tumors. In some embodiments, the tumor, or portion thereof, is a primary tumor. In some embodiments, the tumor is metastases. In some embodiments of the invention, the tumor is a human tumor. As contemplated by this invention, tumor, or portion thereof, may be derived from a cancer patient undergoing anti-cancer therapy, e.g. surgery, chemotherapy, radiation therapy, antibody therapy, immunotherapy, or any combination thereof. In other embodiments, the tumor, or portion thereof, is derived from a patient who has not undergone anti-cancer therapy.

In the humanized mouse model of the invention, one or more human tumor xenografts can be introduced, for example, subcutaneously implanted, by any suitable method known in the art. Methods for implanting a tumor xenograft in a mouse are well known in the art and fully described in Morton and Houghton, Nature Protocols, 2, 247 (Feb. 22, 2007), US Patent application publication US20140109246 A1 and PCT patent application publications WO 2008/143795 and WO 2008/140751, all of which are incorporated by reference herein in their entirety. The tumor or portion thereof may be implanted orthotopically, or at the same site in the recipient mammal as the origin of the tumor. Thus, for example, a kidney tumor may be implanted in the kidney of the recipient mammal. The tumor may also be implanted heterotopically, or in a location that is different from where tumor was derived, for example, and in a preferred embodiment in the flank of the recipient mammal. This invention also provides for implantation of multiple portions of the same tumor in the same mammal, for example both orthotopically and heterotopically. In another embodiment, the portions of the same tumor may be implanted into several individual mammals, all or some of which comprise a subject's or a patient's immune system established as described above. In one embodiment, the tumor, or fragment thereof, is implanted into 2 recipient mammals. In another embodiment, the tumor, or fragment thereof, is implanted into 3 recipient mammals. In another embodiment, the tumor, or fragment thereof, is implanted into 4 recipient mammals. In another embodiment, the tumor, or fragment thereof, is implanted into 5 recipient mammals. In another embodiment, the tumor, or fragment thereof, is implanted into more than 5 recipient mammals. The tumor, or portion thereof, can be removed from the subject and implanted directly into the recipient mammal. The tumor may also be cut into small pieces prior to implantation of each piece into recipient mammal or mammals. In one embodiment, the tumor is cut into 5 mm³ pieces prior to implantation. In another embodiment, the tumor is cut into 10 mm³ pieces prior to implantation. In another embodiment, the tumor is cut into 15 mm³ pieces prior to implantation. In another embodiment, the tumor is cut into 20 mm³ pieces prior to implantation. In another embodiment, the tumor is cut into 25 mm³ pieces prior to implantation. In another embodiment, the tumor is cut into 30 mm³ pieces prior to implantation. In another embodiment, the tumor is cut into 5-30 mm³ pieces prior to implantation. In another embodiment, the tumor is cut into 10-25 mm³ pieces prior to implantation. In another embodiment, the tumor is cut into 15-20 mm³ pieces prior to implantation. In another embodiment, the tumor is cut into 10-30 mm³ pieces prior to implantation. In another embodiment, the tumor is cut into 15-30 mm³ pieces prior to implantation. In another embodiment, the tumor is cut into 20-30 mm³ pieces prior to implantation. In another embodiment, the tumor is cut into 25-30 mm³ pieces prior to implantation. In another embodiment, the tumor is cut into 5-10 mm³ pieces prior to implantation. In another embodiment, the tumor is cut into 5-15 mm³ pieces prior to implantation. In another embodiment, the tumor is cut into 5-20 mm³ pieces prior to implantation. In another embodiment, the tumor is cut into 15-20 mm³ pieces prior to implantation. In another embodiment, the tumor is cut into 10-25 mm³ pieces prior to implantation. In another embodiment, the tumor is cut into 15-25 mm³ pieces prior to implantation. In another embodiment, the tumor is cut into 20-25 mm³ pieces prior to implantation.

Depending on a disease treatment, a suitable tumor graft can be used. In one example, the human tumor xenograft is a melanoma tumor graft. In another example, the human tumor xenograft is a colorectal tumor graft. In another example, the human tumor xenograft is a breast tumor graft. In another example, the human tumor xenograft is a lung tumor graft. In another example, the human tumor xenograft is a xenograft of a human mesenchymal chrondrosarcoma. In another example, the human tumor xenograft is a xenograft of a human leiomyosarcoma. In another example, the human tumor xenograft is a xenograft of a human non-small cell lung cancer. In another example, the tumor xenograft may be a graft of any tumor known in the art.

As contemplated by this invention the tumor may be washed prior to implantation into the recipient mammal. Washing solutions comprise saline, serum-free culture medium or any other solution that may be deemed suitable by a skilled artisan. In another embodiment, the tumors are incubated in a culture medium for one or two days prior to implantation. The incubation conditions may be selected to prevent replication of the tumor cells during incubation. In a preferred embodiment, the solid tumor is not dissociated prior to implantation. Implanting a non-dissociated tumor is important, since it preserves the non-cancerous components within the tumor, including, but not limited to, B cell, T cell, NK cells, macrophage, myofibroblasts, fibroblasts, endothelial cells, blood vessels, and/or lymph vessels.

The invention also provides for monitoring of the tumor growth after implantation. The methods of tumor growth monitoring are well known in the art. Suitable methods of monitoring tumor growth comprised analysis of size of the implanted tumor and analysis of cancer stem cells (CSCs), for example by FACS, for CD44⁺, CD24⁺ cells and/or for ADLH⁺ cells.

The present invention further provides for testing treatments after tumor implant has been established. A cancer tissue can be considered “established” after it has been given an appropriate amount of time to develop in the animal after inoculation of the tissue into the animal. In some embodiments, the tissue can be considered to be “established” after it has developed into a tissue having a size ranging from about 100 mm³ to about 300 mm³. In some embodiment, the tissue can be considered to be “established” after it has developed into a tissue having a size ranging from about 50 mm³ to about 500 mm³, from about 125 mm³ to about 250 mm³, from about 75 mm³ to about 400 mm³, or any range therein.

After implantation of tumor grafts, phenotypic stability of the tumor can be evaluated. In one embodiment, after implantation of tumor grafts, engraftment and growth rates can be evaluated. In a particular embodiment, the mouse model of the invention exhibits phenotypic stability of the tumor.

In another example, the invention provides for a method of testing a cancer treatment in the background of the subject's immune system. The method of cancer treatment testing generally comprises the steps of establishing the subject's immune in a non-human mammal as described above; introducing a heterologous tumor from the subject into said non-human mammal; administering a test treatment to said non-human mammal; and evaluating the effect of said treatment in said non-human mammal.

In another aspect, provided herein is a method for identifying an effective therapeutic regimen for treating a tumor in a patient. The treatments that can be tested in the subject's genetic background comprise pharmacotherapy, chemotherapy, radiation therapy, antibody therapy, immunotherapy or any combination thereof. The method may include the steps of providing a humanized mouse having a human tumor xenograft of the invention; testing one or more therapeutic regimens (e.g., therapeutic agents) to evaluate the effect of said regimens on tumor growth inhibition in said mouse; and identifying an effective therapeutic regimen to treat said patient. The therapeutic regimen may include any suitable type of therapeutic treatments that need to be evaluated on tumor growth. In a particular embodiment, the therapeutic regimen is a therapeutic agent. Examples of a therapeutic agent include, but not limited to, small molecule compounds and large molecules (e.g., antibodies).

Various chemotherapeutic agents can be successfully tested singly or in combination in the humanized xenograft mouse model of the invention. Non-limiting examples of such agents include, but not limited to, the following agents. Docetaxel (TAXOTERE) is a member of the taxane class of chemotherapy drugs, and is a semi-synthetic analogue of paclitaxel (Taxol®), an extract from the rare Pacific yew tree Taxus brevifolia. Sorafenib (NEXAVAR) is a small molecular inhibitor of Raf kinase, PDGF (platelet-derived growth factor) and VEGF receptor kinase. Sunitinib (SUTENT) is a small molecule receptor tyrosine kinase inhibitor. ABI-007 belongs to the family of drugs called mitotic inhibitors. It is also called nanoparticle paclitaxel, protein-bound paclitaxel, paclitaxel (Albumin-Stabilized Nanoparticle Formulation), and Abraxane. Paclitaxel (TAXOL) is an anti-cancer taxane drug isolated the compound from the bark of the Pacific yew tree, Taxus brevifolia. Bevacizumab (AVASTIN) is a monoclonal antibody that works by attaching to and inhibiting the action of vascular endothelial growth factor (VEGF) in laboratory experiments. Sirolimus (RAPAMUNE) is an immunosuppressive agent. Bortezomib (VELCADE), gemcitabine (GEMZAR), irinotecan (CAMPTO), ifosfamide (MITOXANA), and fludarabine (FLUDARA) are other examples of chemotherapeutic agents used singly or in combination in cancer therapy.

In a particular embodiment, the therapeutic agents are molecules targeting immune checkpoints.

In another aspect, provided herein is a method for treating a tumor in a subject (e.g., human patient), the method comprising: providing a humanized mouse comprising a human tumor xenograft; testing one or more therapeutic agents to evaluate the effect of said agents on tumor growth inhibition in said mouse; identifying an effective therapeutic agent; and treating said tumor in said subject.

The present invention further provides for a method of selecting candidates for a clinical trial, wherein a candidate's immune system is established in a non-human mammal as described above, and subsequently a prospective treatment is administered to said mammal. Once the prospective treatment has been administered the immune response to said treatment can be evaluated, allowing for prediction of undesirable immune system-based side effects in a candidate. Subsequently the candidates whose immune system established in a non-human animal displayed negative reaction to the prospective treatment can be excluded from clinical trial.

The term “about” as used herein means in quantitative terms plus or minus 5%, or in another embodiment plus or minus 10%, or in another embodiment plus or minus 15%, or in another embodiment plus or minus 20%.

It will be understood by the skilled artisan that the term “administering” encompasses bringing a subject in contact with a composition of the present invention. Compositions may be administered by any method known to a person skilled in the art, such as parenterally, paracancerally, transmucosally, transdermally, intramuscularly, intravenously, intra-dermally, subcutaneously, intra-peritonealy, intra-ventricularly, intra-cranially, intra-vaginally or intra-tumorally. In a preferred embodiment, compositions may be administered by intravenous, intra-arterial, or intra-muscular injection of a liquid preparation. Suitable liquid formulations include solutions, suspensions, dispersions, emulsions, oils and the like. In one embodiment, the compositions are administered intravenously and are thus formulated in a form suitable for intravenous administration. In another embodiment, the compositions are administered intra-arterially and are thus formulated in a form suitable for intra-arterial administration. In another embodiment, the compositions are administered intra-muscularly and are thus formulated in a form suitable for intra-muscular administration. In a particularly preferred embodiment the compositions are administered via intravenous injection.

As used herein, the terms “treat” and “treatment” refer to therapeutic treatment, including prophylactic or preventative measures, wherein the object is to prevent or slow down (lessen) an undesired physiological change associated with a disease or condition. Beneficial or desired clinical results include, but are not limited to, curing, suppressing, inhibiting, preventing, reducing the severity of, delaying the onset of, reducing symptoms associated with the disease, disorder or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable, or a combination thereof. “Treatment” can also mean prolonging survival as compared to expected survival if not receiving treatment. Those in need of treatment include those already with the disease or condition as well as those prone to having the disease or condition or those in which the disease or condition is to be prevented. In the context of cancer, the terms “treat” and “treatment” refer inter alia to inhibiting tumor growth, delaying progression, expediting remission, inducing remission, augmenting remission, speeding recovery, increasing efficacy of or decreasing resistance to alternative therapeutics, or a combination thereof.

As used herein, the term “treating” refers to curing a disease. In another embodiment, “treating” refers to preventing a disease. In another embodiment, “treating” refers to reducing the incidence of a disease. In another embodiment, “treating” refers to ameliorating symptoms of a disease. In another embodiment, “treating” refers to increasing performance free survival or overall survival of a patient. In another embodiment, “treating” refers to stabilizing the progression of a disease. In another embodiment, “treating” refers to inducing remission. In another embodiment, “treating” refers to slowing the progression of a disease. The terms “reducing”, “suppressing” and “inhibiting” refer to lessening or decreasing.

The present invention also provides for a pharmaceutical composition comprising leukocytes isolated according to the methods described above. The availability of a pharmaceutical composition comprising large numbers of leukocytes has numerous applications in the cancer patients who may frequently suffer immunodeficiency due to age, anti-cancer therapies (e.g. chemotherapy or radiation therapy), immunosuppressive drug treatment or infection. In addition such composition can be used in treatment of relapsed cancer or metastatic disease that originated from the primary tumor that was the original source of leukocytes.

As used herein the term “pharmaceutical composition” encompasses a therapeutically effective amount of the active ingredient or ingredients tumor associated leukocytes with a pharmaceutically acceptable carrier or diluent.

A “therapeutically effective amount”, in reference to the treatment of tumor, refers to an amount capable of invoking one or more of the following effects: (1) inhibition, to some extent, of tumor growth, including, slowing down and complete growth arrest; (2) reduction in the number of tumor cells; (3) reduction in tumor size; (4) inhibition (i.e., reduction, slowing down or complete stopping) of tumor cell infiltration into peripheral organs; (5) inhibition (i.e., reduction, slowing down or complete stopping) of metastasis; (6) enhancement of anti-tumor immune response, which may, but does not have to, result in the regression or rejection of the tumor; and/or (7) relief, to some extent, of one or more symptoms associated with the disorder. A “therapeutically effective amount” of tumor-associated leukocytes provided herein for purposes of treatment of tumor may be determined empirically and in a routine manner.

The mouse model of the invention can be used to treat any cancer/tumor. Examples of cancers/tumors which may be treated include, but not limited to, a melanoma, a colorectal cancer, a breast cancer (including HER2+ and metastatic), a lung cancer. Additional examples of cancers/tumors which may be treated include, but not limited to, a bladder cancer, a prostate cancer, an ovarian cancer, and a gastrointestinal cancer. Examples of a lung cancer include, but are not limited to a small cell lung cancer (SCLC) or a non-small cell lung cancer (NSCLC).

Cancers to be treated may include primary tumors and secondary or metastatic tumors (including those metastasized from lung, breast, or prostate), as well as recurrent or refractory tumors. Recurrent tumors encompass tumors that appear to be inhibited by treatment, but recur up to five years, sometimes up to ten years or longer after treatment is discontinued. Refractory tumors are tumors that have failed to respond or are resistant to treatment with one or more conventional therapies for the particular tumor type. Refractory tumors include those that are hormone-refractory (e.g., androgen-independent prostate cancer; or hormone-refractory breast cancer, such as breast cancer that is refractory to tamoxifen); those that are refractory to treatment with one or more chemotherapeutic agents; those that are refractory to radiation; and those that are refractory to combinations of chemotherapy and radiation, chemotherapy and hormone therapy, or hormone therapy and radiation.

Therapy may be “first-line”, i.e., as an initial treatment in patients who have had no prior anti-cancer treatments, either alone or in combination with other treatments; or “second-line”, as a treatment in patients who have had one prior anti-cancer treatment regimen, either alone or in combination with other treatments; or as “third-line,” “fourth-line,” etc. treatments, either alone or in combination with other treatments.

Therapy may also be given to patients who have had previous treatments which have been partially successful but are intolerant to the particular treatment. Therapy may also be given as an adjuvant treatment, i.e., to prevent reoccurrence of cancer in patients with no currently detectable disease or after surgical removal of tumor.

The mouse model of the invention can also be used for providing a personalized treatment to treat a tumor in a patient. Accordingly, in another aspect, provided herein is a method for providing a personalized treatment to treat a tumor in a patient, the method comprising: providing a humanized mouse comprising a tumor xenograft obtained from said patient; testing one or more therapeutic agents to evaluate the effect of said agents on tumor growth inhibition in said mouse; identifying an effective therapeutic agent; and treating said tumor in said patient, thereby providing a personalized treatment to treat said tumor in said patient.

The term “TumorGraft,” as used herein, may refer to a human tumor xenograft that can be implanted in a mouse model.

The term “ImmunoGraft,” as used herein, may refer to a humanized mouse comprising a human tumor xenograft, wherein said xenograft is capable of propagating in said mouse, and wherein said mouse is an immune-compromised or immune-deficient mouse reconstituted with a human immune system.

The term “comprise” or grammatical forms thereof, refers to the inclusion of the indicated active agent, such as the tumor-associated leukocytes of this invention, as well as inclusion of other active agents, such as an antibody or functional fragment thereof, and pharmaceutically acceptable carriers, excipients, emollients, stabilizers, etc., as are known in the pharmaceutical industry. In some embodiments, the term “consisting essentially of” refers to a composition, whose only active ingredient is the indicated active ingredient, however, other compounds may be included which are for stabilizing, preserving, etc. the formulation, but are not involved directly in the therapeutic effect of the indicated active ingredient. In some embodiments, the term “consisting essentially of” may refer to components, which exert a therapeutic effect via a mechanism distinct from that of the indicated active ingredient. In some embodiments, the term “consisting essentially of” may refer to components, which exert a therapeutic effect and belong to a class of compounds distinct from that of the indicated active ingredient. In some embodiments, the term “consisting essentially of” may refer to components, which exert a therapeutic effect and may be distinct from that of the indicated active ingredient, by acting via a different mechanism of action, for example. In some embodiments, the term “consisting essentially of” may refer to components which facilitate the release of the active ingredient. In some embodiments, the term “consisting” refers to a composition, which contains the active ingredient and a pharmaceutically acceptable carrier or excipient.

As used herein, the singular form “a,” “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein, the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

Any patent, patent application publication, or scientific publication, cited herein, is incorporated by reference herein in its entirety.

In the following examples, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Thus these examples should in no way be construed, as limiting the broad scope of the invention.

### EXAMPLES

### Example 1

### A Humanized Mouse Model for Preclinical Testing of Monoclonal Antibodies Targeting Immune Checkpoints

### Materials and Methods

Thirty-nine melanoma, colorectal, breast and lung TumorGrafts were characterized with respect to HLA expression and mutation status, as well as expression of the negative immune regulator, PD-L1, which is targeted by therapeutic antibodies currently in clinical trial. Immune-compromised NOG (PrkdcscidIl2rgtm1Sug) mice were reconstituted with human CD34+ cells and the animals monitored for cell engraftment and expansion. Humanized and non-humanized NOG animals were subcutaneously implanted with tumor fragments and engraftment and growth rates were compared between the two groups.

### Results

As early as 6 weeks after humanization was initiated, mature human CD45+ cells could be detected in the circulation of humanized animals. We found at least 70% of reconstituted animals had a >15% hCD45+ cells in the peripheral blood 10 weeks post hCD34+ reconstitution. Of the hCD45+ cells present in the peripheral circulation of humanized animals, 19% were hCD3+ (T cells), the majority (67%) being hCD4+ helper T cells, with 25% being hCD8+. Although still early in the growth phase, tumor volumes in humanized animals are comparable to those in non-humanized animals.

Our study has demonstrated that combining humanized mice with Champions TumorGrafts can generate a novel and unique preclinical model. The ImmunoGraft will allow direct assessment of immuno-modulatory agents on tumor growth and progression in a platform more reflective of the human environment. It will also facilitate research examining the critical interplay between tumors and the immune system, leading to identification of additional drug targets.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

### Example 2

### Procedure for Adoptive Transfer of Splenocytes of Humanized Mice

### Leukocyte Expansion

Human leukocytes were obtained from spleens of humanized mice and expanded in vivo. The passaged cells were found to be viable and to have conserved the effector memory phenotype of donor cells as seen through the presence of CD45RO⁺, CD11a⁺, CXCR3⁺, CD44⁺, CD69⁻, CD62L⁻, CD25⁻ markers.

### Experimental Design

Spleens were collected from immunografted mice (minimum of 6 weeks post human immune reconstitution). Splenocytes were prepared using standard protocols. Briefly, mice spleens were cut into small pieces and pressed through a 100 μm cell strainer. Splenocytes were next washed with sterile PBS twice and an aliquot was tested for cell viability and quantification. Cells were suspended in sterile PBS at a concentration of ≦2.5 million cells per 100 uL and a max of 200 uL will be intravenously administered to each mouse. Each splenocyte preparation allows for the engraftment of 5 to 10 NOG (Prkdc^{scid}Il2rg^{tm1Sug}) mice. Aseptic technique was observed during this entire procedure. Splenocytes can alternatively be cryopreserved in DMSO stocks for later use.

### Analysis

Immunophenotyping by flow cytometry analysis on peripheral blood of mice was performed 12 weeks after splenocyte reconstitution to identify population levels of CD45, CD3, CD19 human markers.

### Results

In average, 80% of viable cells were human CD45 cells and of these, 30-46% were human CD3 (T cells) and 36-60% human CD19 (B-cells), 12 weeks post reconstitution (FIG. 3)

Further analyses shown that the fraction CD45 cells increased with the incubation time comprising on average, 14.7%, 32% and 60.5% of viable cells at 3, 6 and 9 weeks post reconstitution, respectively (FIG. 4A). After nine weeks of incubation robust levels of CD3 T-cells, and CD19 B-cells were also observed (FIG. 4 B).

### CONCLUSION

The authors conclude that transferred splenocytes from humanized mice can be expanded in new mice, are functional and are phenotypically indistinguishable from donor T cells.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

1-69. (canceled)

70. A humanized mouse, reconstituted with human CD34+ cells, said mouse comprising a human patient's tumor cell xenograft, wherein said xenograft is capable of propagating in said mouse.

71. The mouse of claim 70, wherein said mouse is an immune-compromised mouse reconstituted with human CD34+ cells or an immune-deficient nude mouse reconstituted with human CD34+ cells.

72. The mouse of claim 70 wherein said mouse is a Non-Obese Diabetic (NOD) Shi-Scid IL-2R γ^{null} (NOG) mouse.

73. The mouse of claim 72, wherein, after a pre-determined time of reconstitution, said mouse is capable of providing mature human CD45+ cells.

74. The mouse of claim 73, wherein said mouse comprises hCD3+, hCD4+, and hCD8+ cells.

75. The mouse of claim 70, wherein said human tumor xenograft is a melanoma tumor graft, a colorectal tumor graft, a breast tumor graft, is a lung tumor graft, human mesenchymal chrondrosarcoma tumor graft, of a human leiomyosarcoma tumor graft, or a human non-small cell lung cancer tumor graft.

76. A method for producing humanized mouse model, the method comprising:
reconstituting said mouse with human CD34+ cells; and
subcutaneously implanting a human tumor xenograft in said mouse,
optionally confirming the phenotypic stability of the tumor,
thereby producing said humanized mouse model.

77. A method for identifying an effective therapeutic regimen for treating a tumor in a patient, the method comprising:
providing a humanized mouse comprising a human tumor xenograft, wherein said xenograft is capable of propagating in said mouse, and wherein said mouse is an immune-compromised mouse reconstituted with a human immune system or an immune-deficient nude mouse reconstituted with a human immune system;
testing one or more therapeutic agents to evaluate the effect of said agents on tumor growth inhibition in said mouse; and
identifying an effective therapeutic agent to treat said patient.

78. The method of claim 77, wherein said mouse is a Non-Obese Diabetic (NOD) Shi-Scid IL-2R γ^{null} (NOG) mouse.

79. The method of claim 77, wherein said mouse is reconstituted with human CD34+ cells.

80. The method of claim 79, wherein, after a pre-determined time of reconstitution, said mouse is capable of providing mature human CD45+ cells.

81. The method of claim 80, wherein said mouse comprises hCD3+, hCD4+, and hCD8+ cells.

82. The method of claim 77, wherein said human tumor xenograft is a melanoma tumor graft, a colorectal tumor graft, a breast tumor graft, a lung tumor graft, a xenograft of a human mesenchymal chrondrosarcoma, a xenograft of a human leiomyosarcoma, or a xenograft of a human non-small cell lung cancer.

83. A method for treating a tumor in a patient, the method comprising:
providing a humanized mouse comprising a human tumor xenograft, wherein said xenograft is capable of propagating in said mouse;
testing one or more therapeutic agents to evaluate the effect of said agents on tumor growth inhibition in said mouse;
identifying an effective therapeutic agent; and
treating said tumor in said patient.

84. A method for providing a personalized treatment to treat a tumor in a patient, the method comprising:
providing a humanized mouse comprising a tumor xenograft obtained from said patient, wherein said xenograft is capable of propagating in said mouse;
testing one or more therapeutic agents to evaluate the effect of said agents on tumor growth inhibition in said mouse;
identifying an effective therapeutic agent; and
treating said tumor in said patient,
thereby providing a personalized treatment to treat said tumor in said patient.

85. A method for establishing a human immune system in a non-human mammal, the method comprising: providing an immunodeficient non-human mammal; injecting said mammal with a composition, said composition comprising human CD34+ progenitor cells or splenocytes isolated from another non-human mammal, wherein said another non-human mammal is a humanized non-human mammal, wherein said isolated splenocytes comprise human immune cells, and wherein said non-human mammal is mouse, rat, pig, rabbit, or guinea pig.

86. The method of claim 85, wherein said humanized non-human mammal is a mammal comprising a human immune system established through injection of a composition comprising human CD34+ progenitor cells, and wherein said humanized non-human mammal is mouse, rat, pig, rabbit, or guinea pig.

87. The method of claim 85, wherein said CD34+ progenitor cells are from human umbilical cord blood, human fetal liver, or a human subject's bone marrow.

88. The established human immune system of claim 86, wherein said system in said non-human mammal comprises human leukocytes, said leucocytes comprising at least about 20% human CD45+ cells.

89. The model of claim 89, wherein said human CD45+ cells in said non-human mammal comprise at least about 5% human CD3+ T cells, at least about 5% human CD19+ B cells, or at least about 1% human CD56+ NK cells.

90. The method of claim 86 further comprising the optional steps of:
sorting said isolated splenocytes to select for a human marker prior to injection into a non-human mammal, wherein said human marker is CD45+, CD3+, CD19+, or CD56+;
propagating said isolated splenocytes in vitro prior to injection into a recipient non-human mammal
stimulating said isolated splenocytes with human cytokines prior to injection into a recipient non-human mammal.

91. A method of testing a therapeutic approach, the method comprising: establishing a human immune system in a non-human mammal in accordance with the method of claim 86; testing a therapy in said mammal; and evaluating the effect of said therapy in said mammal, wherein said therapy is immunotherapy, an immune checkpoint blockade therapy, a therapy by monoclonal antibody, a therapy by a small molecule, a therapy targeting immunosuppressive molecules, a therapy by an immunotherapeutic vaccine, or a combination of any of these with a chemotherapy.

92. A method of testing a cancer therapy, the method comprising the steps of:
a. establishing a human immune system in a non-human mammal in accordance with the method of claim 85;
b. introducing a tumor tissue from a patient;
c. administering a cancer therapy to said non-human mammal; and,
d. evaluating the effect of said therapy in said non-human mammal,
wherein said tumor tissue is introduced by subcutaneous engraftment, orthotopically, or by hematogenous route, and wherein the tumor tissue is a sample of a solid tumor selected from head and neck tumor, a brain tumor, an eye tumor, a thyroid tumor, an adrenal tumor, a salivary gland tumor, an esophageal tumor, a gastric tumor, an intestinal tumor, a colon tumor, a lung tumor, a breast tumor, a liver tumor, a pancreas tumor, a kidney tumor, a bladder tumor, a prostate tumor, a muscular tumor, an osseous tumor, a skin tumor, and a stroma/sarcoma tumor.
